(19) United States
(12) Patent Application Publication
(10) Pub. No.: US 2006/0240444 A1
(43) Pub. Date: Oct. 26, 2006

(54) Detection methods

(76) Inventor: Peiguo Chu, Arcadia, CA (US)

Correspondence Address: FISH & RICHARDSON PC, P.O. BOX 1022, MINNEAPOLIS, MN, 55440-1022, US

(21) Appl. No.: 11/111,677

(22) Filed: Apr. 21, 2005

(51) Int. Cl. C12Q 1/68; G01N 33/551

(52) U.S. Cl. 435/6; 436/524

(57) ABSTRACT

A method of detecting one or multiple target molecules in a test sample.

### BACKGROUND

Early detection is important of a molecule associated with a disorder, in particular, a tumor. A tumor is an overgrowth of neoplastic cells arising from normal tissue after gene mutations. During this process, tumor cells express or secrete into body fluids proteins that do not exist in normal tissues or whose levels exceed normal levels.

Early diagnosis is the key to successfully treating a tumor of high prevalence (e.g., breast, prostate, colon, or lung cancer) and a tumor of deep location (i.e., that escapes routine physical examination, such as pancreas and ovary).

A number of technologies (e.g., tumor tissue microarray technology and serum mass spectrophotometry) have been used in malignancy diagnosis. However, they are time consuming, expensive, or both. There is a need for a more rapid and sensitive method.

### SUMMARY

This invention features a method of detecting a target molecule in a test sample. The method requires use of a plurality of beads coated with a coating antibody that specifically binds to a first epitope of a target molecule (e.g., a protein, a nucleic acid, a lipid, or a carbohydrate), and a detection antibody that specifically binds to a second epitope of the target molecule and is labeled with a fluorophore.

To practice the method, one mixes the just-described beads, detection antibody, and a test sample suspected of containing the target molecule; and determines the intensity of the florescence emitted from the fluorophore on the beads upon irradiation of the fluorophore with an excitation light. The test sample is determined to contain the target molecule if the intensity of the florescent emitted from the fluorophore on one of the beads is above a predetermined value. The method can further include determining the number of beads emitting the florescence. Both the intensity of the florescence and the number of the beads can be determined by flow cytometry.

The just-described method can be used to detect a target molecule in a test sample containing a fluid sample taken from a subject. Examples of the fluid sample include blood, plasma, serum, urine, feces extraction, semen, pleural fluid, peritoneal fluid, and cerebrospinal fluid. A fluid sample can also be a homogenate prepared from a solid or semi-solid tissue sample. In the case where the target molecule is associated with a disorder, the method can be used to diagnose the disorder in a subject. That is, the presence of the target molecule in a test sample indicates that the subject has the disorder.

A “target molecule associated with a disorder” refers to any molecules produced by a disease process (e.g., a damage cell or cell death), a diseased cell (e.g., a tumor cell), or a pathogen (e.g., a parasite, a fungus, a bacterium, a virus, or a prion). Examples of such a target molecule includes antigens expressed or secreted by pathogens or diseased cells (e.g., tumor cells). Exemplary tumors include any human neoplasms, such as a prostate tumor, a breast tumor, a colon tumor, a gynecologic tumor, or a pancreatic tumor. As shown in the example section below, presence of an antigen of a tumor in the test sample indicates that the subject has the tumor.

The above-describe method can be used in simultaneously detecting multiple target molecules (e.g., protein, nucleic acid, lipid, or carbohydrate) in a test sample. The method requires use of the following four items:

(i) a plurality of first beads coated with a first coating antibody that specifically binds to a first epitope of a first target molecule,

(ii) a first detection antibody that specifically binds to a second epitope of the first target molecule and is labeled with a first fluorophore,

(iii) a plurality of second beads coated with a second coating antibody that specifically binds to a first epitope of a second target molecule, and

(iv) a second detection antibody that specifically binds to a second epitope of the second target molecule and is labeled with a second fluorophore.

To practice the method, one (1) mixes the first beads, the second beads, the first detection antibody, the second detection antibody, and a test sample suspected of containing the target molecules to form a mixture, and (2) determines the intensity of the florescence emitted from the first or second beads upon irradiation with an excitation light. The test sample is determined to (a) contain the first target molecule if the intensity of the florescence emitted from the first fluorophore on one of the first beads is above a first predetermined value, and (2) contain the second target molecule if the intensity of the florescence emitted from the second fluorophore on one of the second beads is above a second predetermined value.

The method of this invention can be used to detect at least two target molecules. To this end, the first beads and the second beards can be of the same size; and the first fluorophore and the second fluorophore, upon irradiation with an excitation light, emit florescences of different wavelengths. The two different florescences represent the first target and second target molecules, respectively. Alternatively, the first beads and the second beards can be of different sizes, and the first fluorophore and the second fluorophore, upon irradiation with an excitation light, emit florescence of the same wavelength. In this case, the two different bead sizes represent the first and second target molecules, respectively. The method can further include determining the number of first or second beads emitting florescence. The intensity of the florescence and the number of the beads can be determined by flow cytometry.

The method can be used to simultaneously detect different target molecules associated with different disorders (e.g., different antigens expressed by different tumors). The presence of a particular target molecule (e.g., a tumor antigen) in the test sample indicates that the subject has a disorder with which the target molecule is associated with (e.g., a tumor). For example, the first target molecule and the second target molecule can be respectively a first antigen and a second antigen expressed by a tumor, and the presence of both antigens in the body fluid sample indicates that the subject has the tumor.

For detecting more than two (e.g., four) molecules in a sample, one can from a mixture containing, in addition to the above-described items (i)-(iv), the following four items:

(v) a plurality of third beads coated with a third coating antibody that specifically binds to a first epitope of a third target molecule,

(vi) a third detection antibody that specifically binds to a second epitope of the third target molecule and is labeled with a third fluorophore,

(vii) a plurality of fourth beads coated with a fourth coating antibody that specifically binds to a first epitope of a fourth target molecule, and

(viii) a fourth detection antibody that specifically binds to a second epitope of the fourth target molecule and is labeled with a fourth fluorophore.

Corresponding mixing and detection steps can be conducted in the same manner described above. Similarly, the test sample is determined to contain the third target molecule if the intensity of the florescent emitted from the third fluorophore on one of the third beads is above a third predetermined value; and the test sample is determined to contain the fourth target molecule if the intensity of the florescent emitted from the fourth fluorophore on one of the fourth beads is above a fourth predetermined value.

For accurate detection, readouts from four types of target molecules are differentiated. One can achieve such differentiation by using different combinations of bead sizes/florescence wavelengths. In one example, (i) the first beads and the second beads can be of a first size; and the first fluorophore and the second fluorophore, upon irradiation with an excitation light, emit florescence of different wavelengths; and (ii) the third and the fourth beads are of a second size, and the third fluorophore and the fourth fluorophore, upon irradiation with an excitation light, emit florescence of different wavelengths. In another example, (a) the first beads and the second beards are of different sizes; and the first fluorophore and the second fluorophore, upon irradiation with an excitation light, emit florescence of a first wavelength; and (b) the third beads and the fourth beards are of different sizes, and the third fluorophore and the fourth fluorophore, upon irradiation with an excitation light, emit florescence of a second wavelength. In both examples, the resulting 4 size-wavelength combinations (4=2 sizes×2 wavelengths) represent the four different types of target molecules. As such, one can determine the number of first, second, third, or forth beads emitting florescences and the intensities of the florescences by, e.g., flow cytometry.

When this method is used for diagnosis, each target molecule is associated with a disorder. The presence of the target molecule in a test sample from a subject indicates that the subject has the disorder. For detecting tumors, each target molecule is an antigen expressed by a tumor, and the presence of the antigen in the test sample indicates that the subject has the tumor. In a preferred embodiment, the first target molecule and the second target molecule are respectively a first antigen and a second antigen expressed by a first tumor; and the third target molecule and the fourth target molecule are respectively a first antigen and a second antigen expressed by a second tumor. Presence of both of the first and the second antigens in the body fluid sample indicates that the subject has the first tumor. Similarly, presence of both of the third and forth antigens in the body fluid sample indicates that the subject has the second tumor.

The details of one or more embodiments of the invention are set forth in the description below. Other features, objects, and advantages of the invention will be apparent from the description and the claims.

### DETAILED DESCRIPTION

This invention relates to rapid and sensitive methods for detecting one or more target molecules in a test sample. The methods can be used to diagnose human tumors or other disorders.

To practice the method, one can mix (i) a plurality of beads coated with a coating antibody that specifically binds to a first epitope of a target molecule, (ii) a detection antibody that specifically binds to a second epitope of the target molecule and is labeled with a fluorophore, and (iii) a test sample suspected of containing the target molecule. One then determines the intensity of the florescence emitted from the fluorophore on the beads upon irradiation of the fluorophore with an excitation light. If the intensity of the florescent emitted from the fluorophore on one of the beads is above a predetermined value (i.e., a threshold or cutoff value), the test sample is determined to contain the target molecule. A predetermined or threshold value can be obtained by various suitable methods. For example, one can incubate the just-described coated beads and a detection antibody with a control sample that is free of the target molecule. The average intensity of florescent emitted from all beads represents a predetermined value.

The above-mentioned beads refer to small discrete particles. They should be essentially homogeneous in size and absorbing ability. They can be made of any suitable materials that allow antibodies to absorb from an aqueous solution. To facilitate separation from a test sample, the materials should be solid and insoluble in a sample of interest (e.g., blood) or in an agent for performing antibody-antigen incubation and detection (e.g., a buffer). They should also be inert to components in the sample and the reagent. To minimize detection backgrounds, the materials also should have no or minimal autofluorescence. Examples of suitable materials include ceramics, glass, and polymers. Suitable polymers include polystyrene, polyesters, polyethers, polyolefins, polyalkylene oxides, polyamides, polyurethanes, polysaccharides, celluloses, polyisoprenes, methylstyrene, acrylic polymers, thoria sol, latex, nylon, Teflon cross-linked dextrans (e.g., Sepharose), and cross-linked micelles. Additional examples include carbon graphited, titanium dioxide, and paramagnetic materials. See, e.g., “Microsphere Detection Guide” from Bangs Laboratories, Fishers Ind.

The beads need not be spherical. Irregular beads may be used. The bead sizes can range from nanometers to millimeters, e.g., 100 nm to 1 mm; preferably within 0.2 μm to 200 μm; and more preferably within 1.0 to about 10 μm.

The above-mentioned coating antibody or detection antibody should be specific and sensitive to a target molecule of interest. An “antibody” refers to an immunoglobulin molecule or immunologically active portion thereof, i.e., an antigen-binding portion. The molecule or its antigen-binding portion includes at least one, and preferably two, heavy (H) chain variable regions, and at least one and preferably two light (L) chain variable regions. Examples of an antigen-binding portion include Fab, F(ab′)2, Fv, scFv (single chain antibody), and domain antibody (Ward, et. al. (1989) Nature, 341, 544). Antibodies described herein can be of any immunoglobulin class including IgG, IgM, IgE, IgA, IgD, and any subclass thereof. The antibodies can be a polyclonal or a monoclonal antibody prepared by standard immunology techniques. Alternatively, they can be recombinantly produced, e.g., produced by phage display or by combinatorial methods.

For detecting a tumor, each antibody specifically binds to an antigen produced by cells of the tumor. Preferably, the antigen is a secretory protein that appears in a body fluid. The coating and detection antibodies should bind to two different epitopes of the target molecule so as to avoid competition. Listed below are examples of tumors, suitable body fluids, and corresponding antigens:

| Tumor | Body fluid | Tumor Antigen |
|---|---|---|
| Prostate | serum, | alpha-methylacyl-CoA racemase |
| carcinoma | urine, | (AMACR, P504S), |
| | prostate | prostate specific antigen |
| | secretion | (PSA), |
| | | prostate specific membrane |
| | | antigen (PSMA), |
| | | prostate stem cell antigen |
| | | (PSCA), |
| | | early prostate carcinoma |
| | | antigen (EPCA) |
| Breast | serum, | cytokeratin 19 (CK19), |
| carcinoma | nipple | gross cystic disease fluid |
| | discharge | protein 15 |
| | | (GCDFP-15), |
| | | mammaglobin |
| Colon cancer | serum | Cytokeratin 20 (CK20), |
| | faces extraction | CDX-2 |
| Gynecologic | serum | CA125 and WT-1 for ovarian |
| | | serous tumor, |
| tumors | | hHCG for choriocarcinoma |
| Lung cancer | serum, | cytokeratin 19, |
| | sputum, | surfactant ApoA |
| | bronchioalveolar | |
| | lavage | |
| Pancreatic | serum | CA19-9, |
| adenocarcinoma | | prostate stem cell antigen |
| | | (PSCA). |

To coat antibodies onto beads, one can used various methods known in the art. See, e.g., Harlow, E. and Lane, D. (1988) Antibodies: A Laboratory Manual, Cold Spring Harbor Laboratory Press, Cold Spring Harbor, N.Y.) and “Microsphere Detection Guide” from Bangs Laboratories, Fishers Ind. To check the quality of large-scale preparation of coated beads, one can use flow cytometry to measure the amount and consistency of the bound antibodies.

Beads coated with an antibody against a target molecule can be paired with another antibody against a different epitope of the molecule. The latter is labeled with a fluorophore, and is referred as a detection antibody. The pair of coating/detection antibodies can be any suitable pair of antibodies against a target of interest. For tumor detection, they can be selected according to the table above. Any art-recognized fluorophores can be used. Examples include rhodamine (RD1), fluorescecin isothicyanate (FITC), R-phycoerythrin (PE), cyanin 5, coerythrin-cyanine 5 (PC5), allophycocyanin (APC), . . . etc. Techniques for labeling antibody are well known in the art.

To detect a target molecule, one can mix and incubate the just-described coated beads and detection antibody with a sample suspected of containing the target molecule. For detecting a target molecule that is associated with a disorder in a subject, a suitable body fluid sample is used. To minimize unspecific binding, tissue or cell debris should be removed from the body fluid sample by standard techniques (e.g., centrifugation) before mixing. The beads, detection antibody, and test sample can be mixed and incubated at the same time or sequentially. For example, the beads and the sample can be mixed and incubated first. The resultant mixture is incubated for time sufficient to allow antigen-antibody binding. Afterwards, the mixture can be analyzed by flow cytometry to examine the fluorescence emitted from each bead.

Flow cytometry instrumentation and techniques are well-known in the art. They can be used in practicing the method described above. In general, flow cytometry relies on the passage of a stream of bead suspension through a light beam and electro-optical sensors in such a manner that only one bead at a time passes through the beam-sensor region. As each bead passes this region, the light beam is perturbed by the bead, and the resulting scattered and fluorescent light are detected. The detected optical signals are used by the instrumentation to identify the subgroup to which each bead belongs, along with the presence and amount of label, so that individual bead results are achieved. Descriptions of instrumentation and methods for flow cytometry can be found in the literature. See, e.g., McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

The above-described method can be used to detect multiple target molecules simultaneously. To this end, a set of parameters are required to differentiate fluorescence readouts corresponding to different target molecules.

One suitable parameter for distinguishing various groups of beads is the bead size. Beads of different sizes generate different forward light scatters (FSC), which can be pick up and recorded by flow cytometry.

Methods are known to manufacture beads of a particular size. Due to manufacture variation, beads in general do not have the exact same size. Instead, they tend to have different diameters within a small range spanning a mean diameter. As a result, when using beads of two or more sizes, one should take care to ensure accurate range differentiation. Criteria for range differentiation by flow cytometry are known to those skilled in art of flow cytometry. For example, the diameter of all beads of a group are within ±5% coefficient of variation (CV) or less of the group mean diameter. On the other hand, the minimum difference between mean diameters of any two beads groups is greater than 5% (e.g., 6%, 8%, 10%, or greater) of the s mean diameter of one group. Preferably, the minimum difference is greater than three times of the standard deviation of the group that has the largest standard deviation.

Another suitable parameter is the wavelength of fluorescence emitted from labeled antibody. By using a plurality of fluorescent emissions at various wavelengths, one can use the wavelength difference to distinguish the bead groups from each other. Again, to ensure accurate differentiation, care must be taken to select fluorophores having no or a minimum spectra overlap.

Other suitable parameters include light scatter, light emission, or combinations of light scatter and emission. Side angle light scatter varies with bead size, granularity, absorbance and surface roughness, while forward angle light scatter is mainly affected by size and refractive index. Thus, varying any of these qualities can serve as a means of distinguishing various groups. Light emission can be varied by labeling fluorescent materials onto detection antibodies and using fluorescent materials that have different fluorescence intensities or that emit fluorescence at different wavelengths as mentioned above, or by varying the amount of fluorescent material labeled.

To simultaneously detect four target molecules, one can select two bead sizes and two fluorophores in the manner listed below to generate four combinations. More specifically, one can coat beads of 3.0 μm in diameter with a first coating antibody (“3.0 μm-CAb1”) and a second coating Ab (“3.0 μm-CAb2”) and beads of 4.0 μm in diameter with a third coating antibody (“4.0 μm-CAb3”) and a forth coating antibody (“4.0 μm-CAb4”). These four coating antibodies specifically bind to four target molecules (“TM1,” “TM2,” “TM3,” and “TM4”), respectively. Also used are four labeled detection antibodies that bind to the four target molecules respectively (“DAb1,” “DAb2,” “DAb3,” and “DAb4”). These four DAbs are labeled with Cy5 or FITC as shown in the table below:

Bead Size-CAb

| | 3.0 μm-CAb1 | 4.0 μm-CAb3 |
|---|---|---|
| Fluorophore-DAb | 3.0 μm-CAb2 | 4.0 μm-CAb4 |
| Cy5-DAb1 | TM1 | |
| Cy5-DAb3 | | TM3 |
| FITC-DAb2 | TM2 | |
| FITC-DAb4 | | TM4 |

According to the above-described approach, one can detect four target molecules simultaneously in one test sample. In one example, TM1 and TM2 are two molecules (e.g., proteins) associated with a first disorder (e.g., breast cancer), and TM3 and TM4 are two molecules associated with a second disorder (e.g., colon cancer). Presence of both TM1 and TM2 in a sample from a subject indicates that the subject has the first disorder. Similarly, presence of both TM3 and TM4 in the sample indicates that the subject has the second disorder. By the same token, one can use, e.g., beads of 3 different sizes and 2 different fluorophores to detect 6 (=3×2) different target molecules or 3 disorders simultaneously.

For standardized detection, a particular bead size can be assigned to a particular type of disorder, such as a tumor. For example, beads of 3.0 μm, 4.0 μm, 5.0 μm, 6.0 um, 7.0 μm in diameter are respectively assigned to prostate cancer, breast cancer, colon cancer, lung cancer, and ovary, and so on. It is known that more than one antigens are preferred or required for detecting one particular type of tumor. To enhance detection accuracy and sensitivity, antibodies respectively against multiple antigens of a tumor can be coated on the same bead of a particular size. The presence of all antigens in a sample from a subject indicates that the subject has the tumor.

The above-described method can also be used to detect a molecule in an environmental sample (e.g., water or soil). Such a method is useful for monitoring environment for, e.g., pathogens or toxins. Examples of the molecule includes agents for biological weapons, such as anthrax, glanders, plague, typhoid, paratyphoid A and B, typhus, smallpox, tularemia, infectious jaundice, gas gangrene, tetanus, cholera, dysentery, scarlet fever, undulant fever, tick encephalitis, whooping cough, diphtheria, pneumonia, venereal diseases, tuberculosis, Salmonella, Ebola, Marburg, Machupo, Junin, and Venezuelan equine encephalitis.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE

Polystyrene or silicone latex beads of 3.0 μm or 6.0 μm in diameter (i.e., 3.0 μm or 6.0 μm beads) are obtained. The 3.0 μm beads are split into two groups and coated respectively with antibodies against prostate carcinoma related antigen PSA and AMACR by a standard method. The 6.0 μm beads are also split into two groups and coated respectively with antibodies against lung cancer specific antigens cytokeratin 19 and surfactant ApoA. Alternatively, each of the 3.0 μm beads is coated with both anti PSA antibody and anti-AMACR antibody; each of the 6.0 μm beads is coated with both anti-cytokeratin 19 antibody and anti-surfactant ApoA antibody.

Briefly, the concentration of each type of bead is adjusted to 1×10⁸ beads/ml. To ensure consistent coating, amounts of coating antibodies are normalized based on the total bead surface areas. Beads and coating antibodies are gently mixed and incubated for 6 hours at 4° C. The beads are then centrifuged at 15,000 g for 5 minutes. The supernatant is removed. Excessive hydrophobic binding sites on the beads are blocked by incubation with whole calf serum at 37° C. for 30 minutes. The beads thus-coated are washed twice and resuspended in a PBS/CS/AZ buffer. The quality of the coated beads are checked by flow cytometry to make sure that the coatings are consistent. A sample from each group is diluted, and the bead number in each group is determined by a flow cytometer.

Detection antibodies are conjugated with different fluorophores by standard techniques. More specifically, the just-described four detection antibodies are labeled with four different fluorophores as shown below:

| Antibody | Fluorophore |
|---|---|
| anti-PAS antibody | FITC |
| anti-AMACR | PE |
| anti-cytokeratin 19 | PE |
| anti-surfactant ApoA | FITC |

To detect the above-mentioned four antigens, 2-5 ml of whole blood is drawn from a subject by venipuncture. Serum is separated from cells within 45 minutes by standard techniques. The antibody coated beads described above are suspended in a PBS/CS/AZ buffer and vortexed to obtain a homogeneous suspension. Equal volumes (e.g., 0.5 ml) of bead suspension and serum are mixed, gently vortexed, and incubated at 4° C. for 30 minutes. The beads are then centrifuged at 15,000 g for 5 minutes and the supernatant removed. The bead pellet is resuspended in the PBS/CS/AZ buffer before being mixed with the fluorescent labeled detection antibodies. The mixture are incubated at 4° C. for 30 minutes with gentle vortex.

Flow cytometry measurements are carried out on a Beckman Coulter Cytomics FC 500 MPL Flow Cytometry System, which can perform automated 5-color analysis using either single or dual laser excitation. Fluorophore-labeled beads, upon excitement by a laser, emit fluoresces. Histogram is then obtained and used to display the number of beads (y axis) versus fluorescence intensity (x axis). Gating is performed to isolate electronically the special group of beads of same size depending on side scatter (SSC) determined by brightness of fluorescence and forward light scatter (FSC) determined by bead size.

Contourgram is also obtained by standard techniques. The contourgram, as shown below, allows one to compute the percentage of bead groups as determine by two fluorescences and two sizes. When the cutoff intensity points (inside quadrant 3) of the two fluorescences are determined, the contourgram can be divided into four quadrants, and percentage of each bead subpopulation can be readily determined.

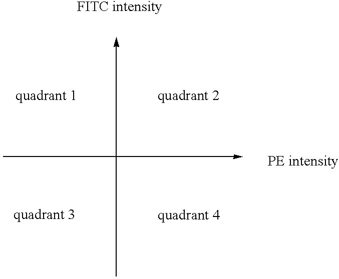

In general, presence of fluorescent beads (either 3.0 μm or 6.0 μm) in quadrant 2 indicates that the subject has a prostate carcinoma or a lung cancer. For example, a presence of 3.0 μm beads in quadrant 2 is diagnostic of a prostate carcinoma. In contrast, if 3.0 um beads are all in the other three quadrants, the subject is determined to be free of a prostate carcinoma since a prostate carcinoma must express both PAS and AMACR. Similarly, presence of 6.0 μm beads in quadrant 2 is diagnostic of a lung cancer; and presence in the other three quadrants suggests otherwise. Presences of both 3.0 μm and 6.0 μm beads in quadrant 2 indicates that the subject has both a prostate carcinoma and a lung cancer. If all fluorescent beads are in quadrant 3, the subject is determined to be free of a prostate carcinoma or a lung cancer.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims

What is claimed is:

1. A method of detecting a target molecule in a test sample, the method comprising
obtaining a plurality of beads coated with a coating antibody that specifically binds to a first epitope of a target molecule, and a detection antibody that specifically binds to a second epitope of the target molecule and is labeled with a fluorophore;
mixing the beads, the detection antibody, and a test sample suspected of containing the target molecule; and
determining the intensity of the florescence emitted from the fluorophore on the beads upon irradiation of the fluorophore with an excitation light,
wherein the test sample is determined to contain the target molecule if the intensity of the florescent emitted from the fluorophore on one of the beads is above a predetermined value.

2. The method of claim 1, wherein the target molecule is a protein, a nucleic acid, a lipid, or a carbohydrate.

3. The method of claim 1, further comprising determining the number of beads emitting the florescence.

4. The method of claim 3, wherein the intensity of the florescence and the number of beads are determined by flow cytometry.

5. The method of claim 1, wherein the test sample contains a fluid sample taken from a subject.

6. The method of claim 5, wherein the fluid sample is a sample of blood, plasma, serum, urine, feces extraction, semen, pleural fluid, peritoneal fluid, or cerebrospinal fluid.

7. The method of claim 5, wherein the target molecule is associated with a disorder, and the presence of the target molecule in the test sample indicates that the subject has the disorder.

8. The method of claim 7, wherein the target molecule is an antigen expressed by a tumor, and the presence of the antigen in the test sample indicates that the subject has the tumor.

9. The method of claim 8, wherein the fluid sample is a sample of blood, plasma, serum, urine, feces extraction, semen, pleural fluid, peritoneal fluid, or cerebrospinal fluid.

10. The method of claim 8, wherein the tumor is a prostate tumor, a breast tumor, a colon tumor, a gynecologic tumor, or a pancreatic tumor.

11. The method of claim 10, further comprising determining the number of beads emitting the florescence.

12. The method of claim 11, wherein the intensity of the florescence and the number of beads are determined by flow cytometry.

13. A method of simultaneously detecting multiple target molecules in a test sample, the method comprising
obtaining (1) a plurality of first beads coated with a first coating antibody that specifically binds to a first epitope of a first target molecule, (2) a first detection antibody that specifically binds to a second epitope of the first target molecule and is labeled with a first fluorophore, (3) a plurality of second beads coated with a second coating antibody that specifically binds to a first epitope of a second target molecule, and (4) a second detection antibody that specifically binds to a second epitope of the second target molecule and is labeled with a second fluorophore;
mixing the first beads, the second beads, the first detection antibody, the second detection antibody, and a test sample suspected of containing the target molecules to form a mixture;
determining the intensity of the florescence emitted from the first or second beads upon irradiation with an excitation light,
wherein the test sample is determined to contain the first target molecule if the intensity of the florescence emitted from the first fluorophore on one of the first beads is above a first predetermined value, and the test sample is determined to contain the second target molecule if the intensity of the florescence emitted from the second fluorophore on one of the second beads is above a second predetermined value.

14. The method of claim 13, wherein the first beads and the second beards are of the same size, and the first fluorophore and the second fluorophore, upon irradiation with an excitation light, emit florescences of different wavelengths.

15. The method of claim 13, wherein the first beads and the second beards are of different sizes, and the first fluorophore and the second fluorophore, upon irradiation with an excitation light, emit florescence of the same wavelength.

16. The method of claim 13, wherein the first or second target molecule is a protein, a nucleic acid, a lipid, or a carbohydrate.

17. The method of claim 13, further comprising determining the number of first or second beads emitting the florescence.

18. The method of claim 17, wherein the intensity of the florescence and the number of beads are determined by flow cytometry.

19. The method of claim 13, wherein the test sample contains a fluid sample taken from a subject.

20. The method of claim 19, wherein the fluid sample is a sample of blood, plasma, serum, urine, feces extraction, semen, pleural fluid, peritoneal fluid, or cerebrospinal fluid.

21. The method of claim 19, wherein each target molecule is associated with a disorder; and the presence of the target molecule in the test sample indicates that the subject has the disorder.

22. The method of claim 21, wherein each target molecule is an antigen expressed by a tumor, and the presence of the antigen in the test sample indicates that the subject has the tumor.

23. The method of claim 22, wherein the fluid sample is a sample of blood, plasma, serum, urine, feces extraction, semen, pleural fluid, peritoneal fluid, or cerebrospinal fluid.

24. The method of claim 22, wherein the tumor is a prostate tumor, a breast tumor, a colon tumor, a gynecologic tumor, or a pancreatic tumor.

25. The method of claim 24, further comprising determining the number of first or second beads emitting the florescence.

26. The method of claim 25, wherein the intensity of the florescence and the number of beads are determined by flow cytometry.

27. The method of claim 26, wherein the first target molecule and the second target molecule are respectively a first antigen and a second antigen expressed by the tumor, and the presence of both antigens in the body fluid sample indicates that the subject has the tumor.

28. The method of claim 13, the mixture further containing
a plurality of third beads coated with a third coating antibody that specifically binds to a first epitope of a third target molecule,
a third detection antibody that specifically binds to a second epitope of the third target molecule and is labeled with a third fluorophore,
a plurality of fourth beads coated with a fourth coating antibody that specifically binds to a first epitope of a fourth target molecule, and
a fourth detection antibody that specifically binds to a second epitope of the fourth target molecule and is labeled with a fourth fluorophore; and
wherein the test sample is determined to contain the third target molecule if the intensity of the florescent emitted from the third fluorophore on one of the third beads is above a third predetermined value; and the test sample is determined to contain the fourth target molecule if the intensity of the florescent emitted from the fourth fluorophore on one of the fourth beads is above a fourth predetermined value.

29. The method of claim 28 wherein
the first beads and the second beads are of a first size, and the first fluorophore and the second fluorophore, upon irradiation with an excitation light, emit florescence of different wavelengths; and
the third and the fourth beads are of a second size, and the third fluorophore and the fourth fluorophore, upon irradiation with an excitation light, emit florescence of different wavelengths.

30. The method of claim 28, wherein
the first beads and the second beards are of different sizes, and the first fluorophore and the second fluorophore, upon irradiation with an excitation light, emit florescence of a first wavelength; and
the third beads and the fourth beards are of different size, and the third fluorophore and the fourth fluorophore, upon irradiation with an excitation light, emit florescence of a second wavelength.

31. The method of claim 28, wherein the first, second, third, or forth target molecule is a protein, a nucleic acid, a lipid, or a carbohydrate.

32. The method of claim 28, further comprising determining the number of first, second, third, or forth beads emitting the florescence.

33. The method of claim 32, wherein the intensity of the florescence and the number of beads are determined by flow cytometry.

34. The method of claim 28, wherein the test sample contains a fluid sample taken from a subject.

35. The method of claim 34, wherein the fluid sample is a sample of blood, plasma, serum, urine, feces extraction, semen, pleural fluid, peritoneal fluid, or cerebrospinal fluid.

36. The method of claim 34, wherein each target molecule is associated with a disorder; and the presence of the target molecule in the test sample indicates that the subject has the disorder.

37. The method of claim 36, wherein each target molecule is an antigen expressed by a tumor, and the presence of the antigen in the test sample indicates that the subject has the tumor.

38. The method of claim 37, wherein the fluid sample is a sample of blood, plasma, serum, urine, feces extraction, semen, pleural fluid, peritoneal fluid, or cerebrospinal fluid.

39. The method of claim 38, wherein the tumor is a prostate tumor, a breast tumor, a colon tumor, a gynecologic tumor, or a pancreatic tumor.

40. The method of claim 39, further comprising determining the number of first, second, third, or forth beads emitting the florescence.

41. The method of claim 40, wherein the intensity of the florescence and the number of beads are determined by flow cytometry.

42. The method of claim 41, wherein
the first target molecule and the second target molecule are respectively a first antigen and a second antigen expressed by a first tumor, and the presence of both antigens in the body fluid sample indicates that the subject has the first tumor; and
the third target molecule and the fourth target molecule are respectively a first antigen and a second antigen expressed by a second tumor, and the presence of both antigens in the body fluid sample indicates that the subject has the second tumor.
